(19) United States
(12) Patent Application Publication
(10) Pub. No.: US 2019/0351060 A1
(43) Pub. Date: Nov. 21, 2019

(54) Rectal Foam Formulations

(71) Applicant: FERRING B.V., Hoofddorp (NL)

(72) Inventor: Crilles Casper Larsen, Mex (CH)

(73) Assignee: FERRING B.V., Hoofddorp (NL)

(21) Appl. No.: 16/470,077

(22) PCT Filed: Dec. 14, 2017
(86) PCT No.: PCT/IB2017/057954

§ 371 Date: Jun. 14, 2019

Related U.S. Application Data
(60) Provisional application No. 62/435,265, filed on Dec. 16, 2016.

(51) Int. Cl. A61K 47/10; A61K 47/38; A61K 47/44; A61K 9/00; A61K 9/12

(52) U.S. Cl. CPC A61K 47/10; A61K 47/38; A61K 9/122; A61K 9/0031; A61K 47/44

(57) ABSTRACT

Described herein are pharmaceutical rectal foam formulations, useful, for example, for the rectal administration of therapeutic agents, such as 5-aminosalicylic acid (5-ASA), as well as methods of making such foam formulations, and therapeutic methods using them.

### CROSS-REFERENCE TO RELATED APPLICATIONS

This application claims priority to U.S. provisional application 62/435,265, filed Dec. 16, 2016, the entire contents of which are incorporated herein by reference.

### FIELD

Described herein are pharmaceutical rectal foam formulations, useful, for example, for the rectal administration of therapeutic agents, such as 5-aminosalicylic acid (5-ASA), as well as methods of making such foam formulations, and therapeutic methods using them.

### BACKGROUND

5-ASA (also known as mesalamine) is an anti-inflammatory drug used to treat inflammatory bowel disease, such as ulcerative colitis and mild-to-moderate Crohn's disease. The activity of 5-ASA against these conditions is primarily local, and so 5-ASA typically is administered by a dosage form and route of administration that will deliver the 5-ASA to the colon, for example. Thus, 5-ASA is available in oral and rectal dosage forms, including rectal suppositories and rectal foam formulations.

A 5-ASA rectal foam formulation has been sold in the UK under the name ASACOL® (Warner Chilcott UK Limited). That product includes sorbitan monooleate, polysorbate 20, emulsifying wax, colloidal anhydrous silica, sodium metabisulphite, disodium edetate, ethylhydroxybenzoate, propylhydroxybenzoate, sodium phosphate dodecahydrate or heptahydrate, sodium acid phosphate, glycerol, Macrogol 300, purified water, propane, iso-butane, and n-butane, and provides 1 g 5-ASA per metered dose.

Another rectal foam formulation is UCERIS® (Salix Pharmaceuticals, Inc.), which contains the active ingredients budesonide, and inactive ingredients cetyl alcohol, citric acid monohydrate, edetate disodium, emulsifying wax, polyoxyl (10) stearyl ether, propylene glycol, and purified water, and propellant n-butane, isobutane, and propane. U.S. Pat. No. 5,914,122 is listed in the Orange Book for UCERIS®, and discloses budesonide solutions with a pH not exceeding 6.0 in which the budesonide is dissolved in a solvent which may be water, an alcohol such as ethanol, isopropanol or propylene glycol, or a water/alcohol mixture. The solutions preferably also contain a stabilizer such as sodium ethylenediaminetetraacetic acid, cyclodextrins or mixtures thereof, and are said to be useful as the active ingredient in a rectal enema or a rectal foam.

However, existing rectal foam formulations could benefit from improvement with regard to the delivery of locally active compounds, such as 5-ASA, to the colon. For example, when administering 5-ASA rectally for delivery to the colon to treat, e.g., ulcerative colitis, an ideal foam would diffuse from the point of rectal administration deep into the colon, expand gradually, expand to a large volume such that it is uniformly distributed internally over the intended area of treatment, and exhibit good retention within the colon.

Thus, there remains a need for rectal foam formulations, including 5-ASA formulations, which exhibit improved properties.

### SUMMARY

Described are pharmaceutical rectal foam formulations, including anhydrous and emulsion rectal foam formulations, useful for the rectal administration of therapeutic agents.

In some embodiments, an anhydrous pharmaceutical rectal foam formulation as described herein comprises a therapeutic agent, such as an aminosalicylate drug or a pharmaceutically acceptable salt or ester thereof, a waxy/lipid component, a solvent/carrier component, an emulsifier/surfactant component, a gum/resin component, and a propellant. Exemplary waxy/lipid components are one or more selected from the group consisting of petrolatum, cetyl alcohol, stearyl alcohol, caprylic/capric triglycerides (CCTs), caprylic/capric glycerides, and sodium stearate. Exemplary solvent/carrier components are one or more selected from the group consisting of polyethylene glycol 400, glycerin, propylene glycol, and mineral oil. Exemplary emulsifier/surfactant components are one or more selected from the group consisting of glyceryl isostearate, emulsifying wax, glyceryl stearate, glyceryl tristearate, sorbitan oleate, polyglyceryl-3 laurate and polyglyceryl-3 diisostearate. Exemplary gum/resin components are one or more selected from the group consisting of methylcellulose, hydroxypropyl cellulose, xanthan gum, pectin, and dextrin.

In some embodiments, the anhydrous formulation includes about 5-15% w/w of the waxy/lipid component. In some embodiments, the anhydrous formulation includes about 55-65% w/w of the solvent/carrier component. In some embodiments, the anhydrous formulation includes about 5-25% w/w of the emulsifier/surfactant component. In some embodiments, the anhydrous formulation includes about 0.1-2% w/w of the gum/resin component.

In specific embodiments, an anhydrous formulation comprises about 20% w/w 5-ASA, about 25% w/w polyethylene glycol 400, about 4% w/w glycerin, about 29.35% w/w propylene glycol, about 2.9% w/w CCTs, about 2.1% w/w emulsifying wax, about 0.6% w/w glyceryl stearate, about 0.8% w/w sorbitan oleate, about 12% w/w petrolatum, about 0.2% w/w xanthan gum, and about 10% w/w propellant A31.

In some embodiments, an oil-in-water emulsion pharmaceutical rectal foam formulation as described herein comprises a therapeutic agent, such as an aminosalicylate drug or a pharmaceutically acceptable salt or ester thereof, a solvent/carrier component comprising water, an emulsifier/surfactant component, a waxy/lipid component, a gum/resin component, and a propellant. Exemplary solvent/carrier components are one or more selected from the group consisting of polyethylene glycol 400, glycerin, propylene glycol and C₁₂-C₁₅ alkyl benzoates. Exemplary emulsifier/surfactant components are one or more selected from the group consisting of polyethylene glycol 75, polyethylene glycol 40 stearate, and sorbitan oleate. Exemplary waxy/lipid components are one or more selected from the group consisting of petrolatum, cetyl alcohol, caprylic/capric triglycerides (CCTs), caprylic/capric glycerides, and stearic acid. Exemplary gum/resin components are one or more selected from the group consisting of hydroxyethyl cellulose and xanthan gum.

In some embodiments, an emulsion formulation includes about 30-50% w/w water and about 4% w/w propylene glycol. In some embodiments, an emulsion formulation includes about 2-25% w/w of the emulsifier/surfactant component. In some embodiments, an emulsion formulation includes about 10-25% w/w of the waxy/lipid component. In some embodiments, an emulsion formulation includes about 0.1-0.2% w/w of the gum/resin component.

In specific embodiments, an emulsion formulation described herein comprises about 20% w/w 5-ASA, about 47.95% w/w water, about 4% w/w propylene glycol, about 1.5% w/w polyethylene glycol 75, about 5% w/w polyethylene glycol 40 stearate, about 0.5% w/w, about 18% w/w petrolatum, about 1.25% w/w cetyl alcohol, about 0.2% w/w xanthan gum, and about 8% w/w propellant A31.

In specific embodiments, the aminosalicylate drug is 5-ASA or a pharmaceutically acceptable salt or ester thereof. In some embodiments, an anhydrous or emulsion formulation comprises about 5 w/w to about 30%w/w 5-ASA.

In some embodiments, a formulation further comprises a propellant. In some embodiments, a formulation comprises about 5-15% w/w propellant, suchas one or more of propellant A-31 and propellant A-46.

In some embodiments, a formulation further comprises a penetration enhancer, such as dimethyl isosorbide.

In some embodiments, a formulation further comprises one or more additional components selected from the group consisting of pH adjusting agents, antioxidants, and preservatives.

Also provided is a foam made from the formulations described herein. In some embodiments, the foam may exhibit a volume of expansion of at least 15 mL in 5 minutes, when tested according to monograph 1105 of the European Pharmacopoeia. In some embodiments, the time to foam collapse in the foam collapse test may be at least 3 minutes. In some embodiments, the time to foam dislodgement in the foam inversion test may be at least 20 minutes. In some embodiments, the foam may exhibit a foam cling of no more than 6 cm/g per 1-2 g of foam over a period of 5 mins. In some embodiments, the foam may exhibits a foam density of at least 0.10 g/mL.

Also provided are methods of making an anhydrous formulation comprising: (a) preparing a first mixture of a solvent/carrier component, a waxy/lipid component and an emulsifier/surfactant component; (b) adding a gum/resin component to the first mixture; (c) adding the therapeutic agent, such as aminosalicylate drug or pharmaceutically acceptable salt or ester thereof, to the mixture (b) to form a final mixture; and (d) combining the final mixture with a propellant.

Also provided are methods of making an emulsion formulation comprising: (a) preparing a first mixture of a solvent/carrier component comprising water and a gum/resin component; (b) preparing a second mixture of an emulsifier/surfactant component and a waxy/lipid component; (b) combining the first and second mixtures to obtain a third mixture; (c) adding the therapeutic agent, such as aminosalicylate drug or pharmaceutically acceptable salt or ester thereof, to the third mixture to form a final mixture; and (d) combining the final mixture with a propellant.

Also provided are methods of administering a therapeutic agent, such as an aminosalicylate drug, to a subject in need thereof, comprising rectally administering a formulation as described herein. In some embodiments, the formulation may comprise 5-ASA and may be administered from an aerosol dispenser dispensing an amount of formulation providing from about 1 g to about 5 g 5-ASA per dose.

Also provided are methods of treating inflammatory bowel disease in a subject in need thereof, comprising rectally administering to the subject in need thereof a formulation as described herein. The inflammatory bowel disease may be selected from ulcerative colitis and Crohn's disease.

Also provided are formulations as described herein, for administering an aminosalicylate drug to a subject in need thereof, or for treating an inflammatory bowel disease, such as ulcerative colitis or Crohn's disease, in a subject in need thereof.

Also provided are uses of formulations as described herein in the preparation of medicaments for administering an aminosalicylate drug to a subject in need thereof, or for treating an inflammatory bowel disease, such as ulcerative colitis or Crohn's disease, in a subject in need thereof.

### BRIEF DESCRIPTION OF THE DRAWINGS

FIG. 1 reports results of ex vivo tests described in Example 5. The left panel shows results of a foam cling test on pig colon tissue placed at a 70° angle. The right panel shows results of a foam inversion test using inverted pig colon tissue.

### DETAILED DESCRIPTION

Described herein are pharmaceutical rectal foam formulations, useful, for example, for the rectal administration of therapeutic agents, such as 5-aminosalicylic acid (5-ASA). In accordance with some embodiments, the foam formulations described herein result in foams with desired properties, including a foam that expands gradually, expands to a large total volume, and/or exhibits good retention within the colon.

Terms and Definitions

Technical and scientific terms used herein have the meanings commonly understood by one of ordinary skill in the art of pharmaceutical formulations to which the present invention pertains, unless otherwise defined. Reference is made herein to various methodologies known to those of ordinary skill in the art. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, the term “about” means that the number or range is not limited to the exact number or range set forth, but encompass values around the recited number or range as will be understood by persons of ordinary skill in the art depending on the context in which the number or range is used. Unless otherwise apparent from the context or convention in the art, “about” means up to plus or minus 10% of the particular term.

As used herein, “subject” denotes any mammal, including humans. For example, a subject may be suffering from or at risk of developing a condition that can be diagnosed, treated or prevented with a drug as described herein, or may be taking a drug for other purposes.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the patient or person herself or himself.

The terms “treat”, “treating” or “treatment”, as used herein, include alleviating, abating or ameliorating a disease or condition or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any therapeutic and/or prophylactic benefit.

As used herein, the phrase “effective amount” refers to a dosage that provides the specific pharmacological effect for which the drug is administered in a subject in need of such treatment. It is emphasized that a therapeutically effective amount will not always be effective in treating the conditions described herein, even though such dosage is deemed to be a therapeutically effective amount by those of skill in the art. For convenience only, exemplary dosages and therapeutically effective amounts are provided below with reference to adult human subjects. Those skilled in the art can adjust such amounts in accordance with standard practices as needed to treat a specific subject and/or condition/disease.

Therapeutic Agents

As noted above, the pharmaceutical rectal foam formulations described herein may include one or more therapeutic agents that can be administered rectally for therapeutic effect. In some embodiments, the therapeutic agent exhibits a local action at one or more levels of the colon, such as an agent with antiinfective/antibiotic, antiinflammatory/antiphlogistic, antispastic, antimeteoric, prokinetic or laxative effect.

The pharmaceutical rectal foam formulations may include a derivative of salicylic acid, such as an aminosalicylate drug, such as 5-ASA, or a pharmaceutically acceptable salt or ester thereof 5-ASA also is known as mesalazine, 5-aminosalicylic acid, 2-hydroxy-5-aminobenzoic acid, 3-carboxy-4-hydroxyaniline, mesalamine, and 5-amino-2-hydroxybenzoic acid, and has the molecular formula C₇H₇NO₃ and a molecular weight of 153.14. It is registered under CAS Registry Number 89-57-6 and Einecs 201-919-1.

Exemplary pharmaceutically acceptable salts include acid addition salts, such as hydrochloride salts.

Typically, rectal foam formulations are formulated with a diagnostically or therapeutically effective amount of one or more therapeutic agents, e.g., an amount effective to exert the intended effect. The amount of therapeutic agent will depend on the formulation being prepared, the amount of formulation dispensed per actuation, the particular therapeutic agent being formulated, the desired effect, and the duration for which the formulation is to provide therapy. In some embodiments, the therapeutic agent may be used in the formulation in an amount of from about 1% w/w to about 50% w/w, including from about 5% w/w to about 40% w/w, about 10% w/w to about 30%w/w, and about 15% w/w to about 25% w/w, based on the total formulation, and amounts between any of these values, including 1% w/w, 5% w/w, 10% w/w, 15% w/w, 20% w/w, 25% w/w, 30% w/w, 35% w/w, 40% w/w, 45% w/w, or 50% w/w.

The amount of therapeutic agent dispensed per actuation of a rectal foam dispenser may be from about 0.1 g to about 10 g, including from about 0.5 g to about 5 g, about 1 g to about 3 g, and about 1 g to about 2 g, and amounts between any of these values.

In specific embodiments, a rectal foam formulation as described herein includes about 5% w/w to about 30% w/w 5-ASA, including about 10% w/w, about 15% w/w, about 20% w/w 5-ASA, about 25% w/w 5-ASA, or about 30% w/w 5-ASA, and amounts between any of these values, and the amount of therapeutic agent dispensed per actuation of a rectal foam dispenser is from about 0.5 g to about 5 g, including from about 1 g to about 2 g, and amounts between any of these values.

Formulations

The pharmaceutical rectal foam formulations described herein include anhydrous formulations and oil-in-water emulsion formulations, as described in more detail below. In addition to one or more therapeutic agents, the anhydrous formulations described herein include a waxy/lipid component, a non-aqueous solvent/carrier component, an emulsifier/surfactant component, a gum/resin component, and a propellant, while the emulsion formulations described herein include a solvent/carrier component including water, an emulsifier/surfactant component, a waxy/lipid component, a gum/resin component, and a propellant.

While not wanting to be bound by any theory, it is believed the combinations of components described herein result in a foam with desired properties, such as a foam that expands gradually, expands to a large total volume, and/or exhibits good retention time, does not collapse immediately upon dispensing, and is stable in terms of emulsion breakdown, separation, gelation or inversion on storage. Further information on the properties of the foams described herein are set forth in more detail below and in the examples.

Anhydrous Formulations

As used here, the term “anhydrous” formulation designates a formulation that comprises less than 5% by weight water. In some embodiments, an anhydrous formulation is prepared without the addition of water, although some water may be present. In some embodiments, an anhydrous formulation is essentially free of water, e.g., contains no more than trace amounts of water.

As noted above, the anhydrous formulations described herein include an active agent, a waxy/lipid component, a solvent/carrier component, an emulsifier/surfactant component, a gum/resin component, and a propellant.

The waxy/lipid component of an anhydrous formulation may comprise one or more of petrolatum, cetyl alcohol, stearyl alcohol, stearic acid, lanolin, hydrous lanolin, lanolin alcohol, paraffin, caprylic/capric triglycerides (CCTs), caprylic/capric glycerides and sodium stearate.

In some embodiments, the waxy/lipid component may be present in an anhydrous formulation in an amount of from about 1% w/w to about 25 w/w, about 2% w/w to about 20% w/w, or about 5% w/w to about 15%w/w, including about 10% w/w, based on the total formulation, and amounts between any of these values, including 1% w/w, 2% w/w, 5% w/w, 10% w/w, 15 w/w, 20% w/w, and 25 w/w. In specific embodiments, an anhydrous 5-ASA formulation may include about 5% w/w to about 15% w/w waxy/lipid components, such as 5% w/w, 10% w/w, or 15%w/w waxy/lipid components.

The solvent/carrier component of an anhydrous formulation may comprise one or more of a polyethylene glycol (such as PEG 400), glycerin, propylene glycol, butylene glycol, ethyl acetate, glycerin, glycofurol, diethyl phthalate, ethanol, C₁₂-C₁₅ alkyl benzoates, dimethyl ether, triacetin, tricaprylin, triethyl citrate, almond oil, peanut oil, safflower oil, sesame oil, sunflower oil, soybean oil, castor oil, mineral oil and light mineral oil.

In some embodiments, the solvent/carrier component may be present in an anhydrous formulation in an amount of from about 30% w/w to about 65% w/w, including from about 40% w/w to about 65% w/w, about 45% w/w to about 65% w/w, or about 50% w/w to about 65% w/w, based on the total formulation, and amounts between any of these values, including 30% w/w, 35% w/w, 40% w/w, 45% w/w, 50% w/w, 55% w/w, 60% w/w and 65% w/w. In specific embodiments, an anhydrous 5-ASA formulation may include about 55% w/w to about 65% w/w solvent/carrier component.

The emulsifier/surfactant component of an anhydrous formulation may include one or more of glyceryl isostearate, anionic and nonionic emulsifying waxes, glyceryl monostearate, glyceryl tristearate, glyceryl monooleate, glyceryl palmitostearate, sorbitan oleate, polyglyceryl-3 laurate, polyglyceryl-3 diisostearate, polyethylene glycol 75, polyethylene glycol 40 stearate, myristyl alcohol, mineral oil alcohols, lanolin alcohols, lecithin, linoleic acid, poloxamers (polyoxyethylene/polyoxypropylene/polyoxyethylene block copolymers, such as Poloxamer 181, 182, and/or 331), polyoxyethylene alkyl ethers, polyethoxylated castor oils (such as Polyoxyl 35 castor oil and/or Polyoxyl 40 hydrogenated castor oil), sorbitan fatty acid esters, polyoxyethylene sorbitan fatty acid esters, polyoxyethylene stearates, polyoxylglycerides, vitamin E polyethylene glycol succinate, propylene glycol alginate, and saponite, In specific embodiments the emulsifier/surfactant component of an anhydrous formulation may comprise one or more components selected from the group consisting of glyceryl isostearate (including Global 4075, which is a blend of glyceryl isostearate and caprylic/capric glycerides), emulsifying wax (e.g., Polawax NF), glyceryl stearate, glyceryl tristearate, sorbitan oleate (e.g., Span 80), polyglyceryl-3 laurate and polyglyceryl-3 diisostearate.

In some embodiments, the emulsifier/surfactant component may be present in an anhydrous formulation in an amount of from about 1% w/w to about 45% w/w, including from about 5% w/w to about 45% w/w, or about 5% w/w to about 35% w/w, or about 5% w/w to about 20% w/w, or about 5% w/w to about 15% w/w, based on the total formulation, and amounts between any of these values, including about 5% w/w, 10% w/w, 15% w/w, and 20% w/w. In specific embodiments, an anhydrous 5-ASA formulation may include about 5% w/w to about 25% w/w, including about 5% w/w to about 10% w/w emulsifier/surfactant component.

The gum/resin component of an anhydrous formulation may comprises one or more components selected from the group consisting of methylcellulose, hydroxypropyl cellulose, hydroxyethyl cellulose, hydroxyethyl methyl cellulose, hypromellose, xanthan gum, guar gum, kaolin, acacia, agar, alginic acid, attapulgite, bentonite, carbomer, carboxymethylcellulose calcium, carboxymethylcellulose sodium, carrageenan, gelatin, pectin, polycarbophil, potassium alginate, sodium alginate, tragacanth, povidone, and dextrin. In specific embodiments, the gum/resin component of an anhydrous formulation includes one or more of hydroxyethyl cellulose and xanthan gum.

In some embodiments, the gum/resin component may be present in an anhydrous formulation in an amount of from about 0.01% w/w to about 5% w/w, including from about 0.1% w/w to about 5% w/w, about 0.1% w/w to about 2% w/w, or about 2% w/w, based on the total formulation, and amounts between any of these values, including 0.1% w/w, 0.5% w/w, 1% w/w, 2% w/w, 3% w/w, 4% w/w, and 5% w/w. In specific embodiments, an anhydrous 5-ASA formulation may include about 0.1% w/w to about 2% w/w gum/resin component, or about 0.2% w/w.

Emulsion Formulations

As noted above, the emulsion formulations described herein include an active agent, a waxy/lipid component, a solvent/carrier component including water, an emulsifier/surfactant component, a gum/resin component, and a propellant.

The waxy/lipid component of an emulsion formulation may comprise one or more of petrolatum, cetyl alcohol, stearyl alcohol, stearic acid, lanolin, caprylic/capric triglycerides (CCTs), caprylic/capric glycerides, hydrous lanolin, lanolin alcohol, paraffin, and sodium stearate. In specific embodiments, a 5-ASA emulsion formulation includes one or more of petrolatum, cetyl alcohol, and stearic acid as a waxy/lipid component.

In some embodiments, the waxy/lipid component may be present in an emulsion formulation in an amount of from about 5% w/w to about 30% w/w, or about 10% w/w to about 25% w/w, including about 10% w/w, about 15% w/w, and about 20% w/w, based on the total formulation, and amounts between any of these values, including 10% w/w, 15% w/w, 20% w/w, and 25% w/w. In specific embodiments, a 5-ASA emulsion formulation may include about 10% w/w to about 25% w/w waxy/lipid components, such as about 20% w/w waxy/lipid components.

The solvent/carrier component of an emulsion formulation includes water and, optionally, another solvent/carrier component, such as one or more water-miscible solvent/carrier components, such as any water-miscible solvent/carrier component listed above with reference to anhydrous formulations, including one or more of a polyethylene glycol (such as PEG 400), glycerin, propylene glycol, butylene glycol, ethyl acetate, glycerin, glycofurol, diethyl phthalate, ethanol, dimethyl ether, triacetin, tricaprylin, triethyl citrate, C₁₂-C₁₅ alkyl benzoates, almond oil, peanut oil, safflower oil, sesame oil, sunflower oil, soybean oil, castor oil, mineral oil and light mineral oil.

In some embodiments, an emulsion formulation comprises water in an amount of from about 20% w/w to about 60% w/w, including from about 30% w/w to about 50 w/w, based on the total formulation, and amounts between any of these values, including 20% w/w, 25% w/w, 30% w/w, 35% w/w, 40% w/w, 45% w/w, 50% w/w, 55% w/w, or 60% w/w. In specific embodiments, a 5-ASA emulsion formulation includes about 40% w/w to about 50% w/w water, such as 40% w/w, 45 w/w, or 50 w/w water.

As noted above, in some embodiments, an emulsion formulation comprises, in addition to water, an additional solvent/carrier component such as one or more of a polyethylene glycol (such as PEG 400), glycerin, and propylene glycol, or any one or more of the other solvent/carrier components set forth above. In accordance with such embodiments, the additional solvent/carrier component may be present in any amount, including from about 0.1% w/w to about 10% w/w, including from about 1.0% w/w to about 5% w/w, including from 1.0% w/w to 5% w/w, based on the total formulation, and amounts between any of these values, including 1.0% w/w, 2.0% w/w, 3.0% w/w, 4.0% w/w, or 5.0% w/w. In specific embodiments, a 5-ASA emulsion formulation includes about 30% w/w to about 50% w/w water, and about 1.0% w/w to about 5% w/w propylene glycol, such as about 4% w/w propylene glycol.

The emulsifier/surfactant component of an emulsion formulation may include one or more of propylene glycol dilaurate, sorbitan trioleate, and polyglyceryl-3 polyricinoleate, polysorbate 20, glyceryl isostearate, anionic and nonionic emulsifying waxes, glyceryl monostearate, glyceryl tristearate, glyceryl monooleate, glyceryl palmitostearate, sorbitan oleate, polyglyceryl-3 laurate, polyglyceryl-3 diisostearate, polyethylene glycol 75, polyethylene glycol 40 stearate, myristyl alcohol, mineral oil alcohols, lanolin alcohols, lecithin, linoleic acid, poloxamers poloxamers (polyoxyethylene/polyoxypropylene/polyoxyethylene block copolymers, such as Poloxamer 181, 182, and/or 331), polyoxyethylene alkyl ethers, polyethoxylated castor oils (such as Polyoxyl 35 castor oil and/or Polyoxyl 40 hydrogenated castor oil), sorbitan fatty acid esters, polyoxyethylene sorbitan fatty acid esters, polyoxyethylene stearates, polyoxylglycerides, vitamin E polyethylene glycol succinate, propylene glycol alginate, and saponite. In specific embodiments the emulsifier/surfactant component of an emulsion formulation may comprise one or more components selected from the group consisting of, glyceryl isostearate (e.g., Global 4075), emulsifying wax (e.g., Polawax NF), glyceryl stearate, glyceryl tristearate, sorbitan oleate (e.g., Span 80), polyglyceryl-3 laurate and polyglyceryl-3 diisostearate. In specific embodiments of emulsion formulations, the emulsifier/surfactant component may comprise one or more of polyethylene glycol 75, polyethylene glycol 40 stearate, and sorbitan oleate.

In some embodiments, an emulsion formulation comprises an emulsifier/surfactant component in an amount of from about 1% w/w to about 25% w/w, including from about 2% w/w to about 20% w/w, based on the total formulation, and amounts between any of these values, including about 2% w/w, 2.5% w/w, 3% w/w, 5% w/w, 10% w/w, 15% w/w, and 20% w/w. In specific embodiments, a 5-ASA emulsion formulation includes about 2% w/w to about 15% w/w emulsifier/surfactant component.

The gum/resin component of an emulsion formulation may comprises one or more components selected from the group consisting of methylcellulose, hydroxypropyl cellulose, hydroxyethyl cellulose, hydroxyethyl methyl cellulose, hypromellose, xanthan gum, guar gum, kaolin, acacia, agar, alginic acid, attapulgite, bentonite, carbomer, carboxymethylcellulose calcium, carboxymethylcellulose sodium, carrageenan, gelatin, pectin, polycarbophil, potassium alginate, sodium alginate, tragacanth, povidone, and dextrin. In specific embodiments, the gum/resin component of an anhydrous formulation includes one or more of hydroxyethyl cellulose and xanthan gum. In specific embodiments, the gum/resin component of an emulsion formulation includes one or more of hydroxyethyl cellulose and xanthan gum.

In some embodiments, the gum/resin component may be present in an anhydrous formulation in an amount of from about 0.01% w/w to about 5% w/w, including from about 0.1% w/w to about 5% w/w, about 0.1% w/w to about 2% w/w, or about 2% w/w, based on the total formulation, and amounts between any of these values, including 0.1% w/w, 0.5% w/w, 1% w/w, 2% w/w, 3% w/w, 4% w/w, and 5% w/w. In specific embodiments, an anhydrous 5-ASA formulation may include about 0.1% w/w to about 2% w/w gum/resin component, or about 0.2% w/w.

Other Components

In addition to the foregoing, the formulations described herein may include one or more other optional components, such as one or more penetration enhancers, one or more pH adjusting agents, one or more antioxidants, one or more preservatives, and/or one or more other components suitable for use in a pharmaceutical rectal foam formulation. In specific embodiments, any optional components used do not substantially impact the therapeutic efficacy of the formulation. Additionally or alternatively, in specific embodiments, any optional components used do not substantially impact the foam properties of the formulation. Optional components, if present, can be incorporated in the formulations in any suitable amount sufficient to have the intended effect of the component without substantially interfering with the desired properties of the compositions, such as their foaming and drug delivery properties. Exemplary components and amounts thereof are provided herein below.

In accordance with any embodiments described herein, a penetration enhancer may be used. In specific embodiments of 5-ASA formulations, the penetration enhancer is or includes dimethyl isosorbide, benzalkonium chloride, cetylpridinium chloride, diethyl sebacate, glycofurol, oleyl alcohol, propylene glycol monolaurate, isopropyl myristate, isopropyl palmitate, sodium lauryl sulfate, ethanol, pentylene glycol, propylene glycol and butylene glycol. In some embodiments, the penetration enhancer may be used in the anhydrous or emulsion formulation in an amount of from about 0.5% w/w to about 5% w/w. In specific embodiments, the penetration enhancer may be used in the anhydrous or emulsion formulation in an amount of from about 1% w/w to about 2.5% w/w. In further specific embodiments, an anhydrous 5-ASA formulation may include about 1% w/w dimethyl isosorbide. In further specific embodiments, a 5-ASA emulsion formulation may include about 2.5% w/w dimethyl isosorbide.

In accordance with any embodiments described herein, a pH adjusting agent may be used. In specific embodiments, the pH adjusting agent may be or include triethanolamine, sodium or potassium hydroxides, sodium or potassium bicarbonates or carbonates, potassium or sodium citrates, diethanolamine, monoethanolamine, or tromethamine. In some embodiments, the pH adjusting agent may be used in the anhydrous or emulsion formulation in an amount of from about 0.1%w/w to about 2% w/w. In further specific embodiments, an anhydrous 5-ASA formulation does not include a pH adjusting agent. In further specific embodiments, a 5-ASA emulsion formulation may include about 0.75% w/w triethanolamine as a pH adjusting agent. In accordance with any embodiments described herein, an antioxidant may be used. In specific embodiments, the antioxidant may be selected from one or more of alpha tocopherol, tocopheryl acetate, ascorbic acid, sodium ascorbate, ascorbyl palmitate, citric acid monohydrate, potassium metabisulfite, sodium metabisulfite, sodium sulfite, sodium thiosulfate, vitamin E polyethylene glycol succinate, propyl gallate, butylated hydroxyanisole (BHA) or butylated hydroxytoluene (BHT). In some embodiments, the antioxidant may be used in the anhydrous or emulsion formulation in an amount of from about 0.01% w/w to about 1% w/w. In further specific embodiments, an anhydrous 5-ASA formulation may include from about 0.3% w/w to about 0.7% w/w antioxidant. In further specific embodiments, a 5-ASA emulsion formulation may include about 0.05% w/w to about 0.2% w/w antioxidant.

In accordance with any embodiments described herein, a preservative may be used. In specific embodiments, the preservative may be selected from one or more of methylparaben, propylparaben, sodium benzoate, tetrasodium EDTA, benzalkonium chloride, benzyl alcohol, cetylpyridinium chloride, chlorhexidine, chlorocresol, imidurea, monothioglycerol, sodium borate, thimerosal, ethylparaben, butylparaben, sorbic acid, benzoic acid, undecanoic acid, and glutanaldehyde.

In some embodiments, the preservative may be used in the anhydrous or emulsion formulation in an amount of from about 0.1%w/w to about 1.0%w/w. In further specific embodiments, an anhydrous 5-ASA formulation may include from about 0.1% w/w to about 0.2% w/w preservative, including about 0.15% w/w preservative. In further specific embodiments, a 5-ASA emulsion formulation may include about 0.2% w/w to about 0.4% w/w preservative.

Propellant

The formulations further include a propellant. In some embodiments, the propellant is a conventional aerosol propellant used in pharmaceutical formulations, such as a hydrocarbon propellant (e.g., propane, pentane, butene, butane, isobutane), a hydrofluorocarbon propellant (such as HFC-134 a), or combinations thereof. Specific examples of suitable propellants for either type of formulation include one or more of propellant A-31(isobutene), propellant A-46 (propane/isobutane), and propellant HFC-134a (tetrafluoroethane), trans-1,3,3,3-tetrafluoroprop-1-ene (such as 1234ze from Honeywell), dimethyl ether (DME), or a combination of two or more thereof. In specific embodiments, the propellant includes one or more of propellant A-31 and propellant A-46. When more than one propellant is used, they may be used in any ratio, including 1:1. In specific embodiments, an anhydrous 5-ASA formulation may include from about 10% w/w to about 25% w/w propellant, including about 10% w/w and about 20% w/w propellant. In further specific embodiments, a 5-ASA emulsion formulation may include from about 10% w/w to about 20% w/w propellant, including about 14% w/w, 15% w/w, and 16% w/w propellant

Exemplary Formulations

Described below are various exemplary anhydrous 5-ASA formulations. In accordance with specific embodiments illustrated in the examples below, an anhydrous 5-ASA formulation comprises about 20% w/w 5-ASA, about 25% w/w polyethylene glycol 400, about 4% w/w glycerin, about 29.35% w/w propylene glycol, about 2.9% w/w CCTs, about 2.1% w/w emulsifying wax, about 0.6% w/w glyceryl stearate, about 0.8% w/w sorbitan oleate, about 12% w/w petrolatum, about 0.2% w/w xanthan gum, and about 10% w/w propellant A31.

Described below are various exemplary 5-ASA emulsion formulations. In accordance with specific embodiments illustrated in the examples below, a 5-ASA emulsion formulation includes about 20% w/w 5-ASA, about 47.95% w/w water, about 4% w/w propylene glycol, about 1.5% w/w polyethylene glycol 75, about 5% w/w polyethylene glycol 40 stearate, about 0.5% w/w, about 18% w/w petrolatum, about 1.25% w/w cetyl alcohol, about 0.2% w/w xanthan gum, and about 8% w/w propellant A31.

Preparation of Foam Formulations

The foam formulations described herein may be prepared by methods known in the art. For example, all of the components except the propellant may be combined and filled into a suitable dispenser, and the propellant may be added thereafter. Typically, the formulation (except propellant) is added to the dispenser, and then the propellant is added and the dispenser is pressurized and sealed. The components (except propellant) may be combined and mixed in any suitable order.

In some embodiments, an anhydrous formulation is made by a process comprising (a) preparing a first mixture of a solvent/carrier component, a waxy/lipid component and an emulsifier/surfactant component; (b) adding a gum/resin component to the first mixture; (c) adding the therapeutic agent to the mixture (b) to form a final mixture; and (d) combining the final mixture with a propellant. In some embodiments, the process further includes adding other optional components at appropriate stages, prior to combining with propellant.

In some embodiments, an emulsion formulation is made by a process comprising (a) preparing a first mixture of a solvent/carrier component comprising water and a gum/resin component; (b) preparing a second mixture of an emulsifier/surfactant component and a waxy/lipid component; (c) combining the first and second mixtures to obtain a third mixture; (d) adding the therapeutic agent to the third mixture to form a final mixture; and (d) combining the final mixture with a propellant. In some embodiments, the process further includes adding other optional components at appropriate stages, prior to combining with propellant.

Dispensers for Foam Formulations

The foam formulations described herein may be provided in any suitable dispenser for a pharmaceutical foam formulation. Typically, the dispenser is adapted to the route of administration, such as e.g., rectal. In some embodiments, the dispenser is a metered dose dispenser, such as a dispenser having a metered dose dispensing valve that dispenses one dose of the formulation at a time. In some embodiments, the dispenser is an aerosol dispenser.

A unit dose will depend on the therapeutic agent being administered, its concentration in the formulation, and the condition being treated, and may vary with other factors, such as the age, weight or condition of the subject. In specific embodiments relating to 5-ASA formulations, a unit dose may provide from about 0.1 g to about 10 g of 5-ASA (or an equivalent amount of another aminosalicylate drug, or a pharmaceutically acceptable salt or ester of 5-ASA or other aminosalicylate drug), including from about 0.5 g to about 5 g, about 1 g to about 3 g, and about 1 g to about 2 g, and amounts between any of these values, including 0.5 g, 1 g, 1.5 g, 2 g, 3 g, 4 g, or 5g of 5-ASA (or an equivalent amount of another aminosalicylate drug, or a pharmaceutically acceptable salt or ester of 5-ASA or other aminosalicylate drug). In some embodiments, a metered dose contains 1 g of 5-ASA, 2 g of 5-ASA, or 4 g of 5-ASA (or an equivalent amount of another aminosalicylate drug, or a pharmaceutically acceptable salt or ester of 5-ASA or other aminosalicylate drug).

Properties of Foam Formulations

Various properties of the foam formulations described herein can be assessed by methods known in the art, such as those set forth in monograph 1105 of the European Pharmacopoeia. For example, one or more of foam expansion, foam cling, foam inversion, foam density, and foam collapse, and can be assessed by methods known in the art.

In accordance with some embodiments, the 5-ASA foam formulations described herein exhibit one or more properties that are improved as compared to the ASACOL® foam formulation.

Foam Expansion

As noted above, when administering 5-ASA rectally for delivery to the colon to treat, e.g., ulcerative colitis, an ideal foam will expand gradually and expand to a large volume such that it is uniformly distributed internally over the intended area of treatment.

Volume of expansion can be assessed as follows, which is based on Eur. Pharm. 1105:

A 72.5 cm×1.3 cm Pyrex 50 mL burette with 1 mL graduations is used as the measuring device. It is attached to a mousse actuator (a Precision Valve, Juno, 1 inch mousse spout, 4 mm stem diameter) by means of a 7.5 cm×5 mm flexible tube. Sample containers are maintained at about 25° C. for at least 24 hours before testing. Samples are shaken vigorously for approximately 30 seconds and 5 to 10 ml of foam is dispensed to waste before attachment to the flexible tube/mousse spout. Once the actuator has been attached securely to the flexible tube, the container is inverted and the actuator is depressed to dispense a sufficient quantity of foam to reach the 40-30 mL burette gradations. The burette stopcock is immediately closed and timing of expansion is begun by calibrated stopwatch. Foam levels (L) are recorded every minute (0-5) for 5 minutes. If the foam level surpasses the 0 mL gradation before the 5 minutes expires, the time taken to reach the 0 mL mark is recorded and noted. The volume of expansion over the test period is calculated the difference between the maximum level and the initial level.

When tested in accordance with this protocol prior to storage, the ASACOL® foam formulation exhibits a volume of expansion of about 5 ml over 5 minutes. In contrast, foam formulations described herein (when tested in accordance with this protocol prior to storage) may exhibit a volume of expansion of greater than 5 ml, such as greater than 10 ml, greater than 15 ml, greater than 20 ml, greater than 25 ml, greater than 30 ml, or greater than 35 ml. In specific embodiments, a foam formulation as described herein exhibits a volume of expansion of at least 15 mL over 5 minutes. In specific embodiments, a foam formulation as described herein exhibits a volume of expansion of at least 20 mL over 5 minutes.

Foam Cling

As noted above, when administering 5-ASA rectally for delivery to the colon to treat, e.g., ulcerative colitis, an ideal foam exhibits good retention within the colon. While not necessarily representative of colonic lumen, foam cling can be measured in vitro or ex vivo as the distance one gram of foam travels down a specific incline (such as a 60° incline) over a certain period of time (such as 5 minutes). (The shorter the distance, the greater the “cling” property.) Improved foam cling properties indicate improved cohesiveness and/or adhesiveness of the foam formulations, at least with respect to the surfaces on which the test is conducted

Foam Cling can be Assessed as Follows:

A 5 mm×28.5 cm×28.5 cm Plexiglas plate is marked horizontally in 1 cm increments 1 to 25 cm. The plate is placed in an aluminum base that has been previously milled to accept and hold the plate at 60° from vertical. Sample containers are maintained at about 25° C. for at least 24 hours before testing. Samples are shaken vigorously for approximately 30 seconds and 5 to 10 ml of foam is dispensed to waste. The container is weighed and that weight is recorded as the initial weight (W₀). Samples are actuated for approximately 2 seconds onto the 2 cm marked line. A calibrated stop watch is used to begin timing immediately (T₀=initial time) for a period no longer than 5 minutes. The container is weighed after dispensing and that weight recorded as the final weight (W_{F}), and weight is calculated by subtracting W₀ from W_{F}). Time is calculated by subtracting T₀ from the final time (T_{F}). Foam cling is calculated as follows:

Foam Cling (cm/g)=(D_{F}-D₀)/(W₀-W_{F})

Foam Cling ((cm/g)/min)=((D_{F}-D₀)/(W₀-W_{F}))/(T_{F} T₀)

When tested in accordance with this protocol prior to storage, the ASACOL® foam formulation exhibits a foam cling of about 7.5 cm/g. In accordance with some embodiments, a foam produced by an either type of formulation as described herein (when tested in accordance with this protocol prior to storage) exhibits a foam cling of less than 7.5 cm/g, such as less than 5 cm/g, less than 3 cm/g, or less than 2 cm/g. 33. In specific embodiments, a foam as described herein exhibits a foam cling of no more than 6 cm/g per 1-2 g of foam.

Foam Inversion

Foam inversion is another measure of foam retention properties, e.g., cohesiveness and/or adhesiveness of the foam formulations.

Foam inversion can be measured as the time elapsed for a foam to dislodge and fall from an inverted glass. A specific protocol is outlined below:

Sample containers are maintained at about 25° C. for at least 24 hours before testing. Samples are shaken vigorously for approximately 30 seconds and 5 to 10 ml of foam are dispensed to waste. Sample container weight is recorded (W₀) and samples are actuated over a 12.1 cm (4¾ inch) diameter circle previously marked on a 30.5 cm×45.7 cm (12 in×18 in) tempered glass plate. Foam is dispensed to cover the area within the delineations of one circle and the sample containers are weighed again (W_{F}). (To compare values between tests, an attempt is made to dispense approximately equal amounts over an equal area, to obtain equal weights per surface area.) The glass plate is inverted so the foam is facing down and time is recorded as T₀. A calibrated stop watch is used to measure the time it takes for the foam to dislodge and fall from the inverted surface (T_{X}).

Foam Inversion (min/g)=T_{X}/(W₀-W_{F})

When tested in accordance with this protocol prior to storage, the ASACOL® foam formulation exhibits a foam inversion time of about 1.6 minutes per about 20 g. In accordance with some embodiments, a foam produced by either type of formulation as described herein (when tested in accordance with this protocol prior to storage) exhibits a foam inversion time greater than that of the ASACOL® foam formulation, such as greater than about 5 minutes, greater than about 10 minutes, greater than about 15 minutes, or greater than about 20 minutes, all for about 20 g of foam.

Foam Density

Foam density is measured as the weight of foam per unit volume.

When tested prior to storage, the ASACOL® foam formulation exhibits a foam density of about 0.067 g/ml. In accordance with some embodiments, a foam produced by an emulsion formulation as described herein (when tested prior to storage) exhibits a foam density comparable to that of the ASACOL® foam, such as a foam density of from about 0.05 g/ml to about 0.07 g/ml. In accordance with some embodiments, a foam produced by an anhydrous formulation as described herein (when tested prior to storage) exhibits a foam density greater than that of the ASACOL® foam, such as a foam density of greater than about 0.067 g/ml, such as at least about 0.08 g/ml, or at least about 0.1 g/ml, including about 0.1 g/ml. In accordance with some embodiments a foam produced by an emulsion formulation as described herein (when tested prior to storage) exhibits a foam density of at least 0.05 g/ml, including about 0.05 g/ml.

Foam Collapse

In the context of administering a foam formulation for transmucosal delivery of an active agent, foam collapse is a measure of how quickly and for how long the active components of the foam formulation will come into contact with mucosa, because once the foam breaks the bulk of the formulation is in direct contact with the mucosal surface, but may limit overall duration of contact time between the mucosa and the formulation. Foam collapse can be measured as the time elapsed (in minutes) from when the foam is placed in a drying oven set at 36-37° C. to when the last observable foam bubble has broken.

When tested in accordance with this protocol prior to storage, the ASACOL® foam formulation exhibits a foam collapse time of greater than 5 minutes. In accordance with some embodiments, a foam produced by an emulsion formulation as described herein (when tested prior to storage) exhibits a foam collapse comparable to that of the ASACOL® foam, such as a foam collapse time of greater than 5 minutes. In accordance with some embodiments, a foam produced by an anhydrous formulation as described herein (when tested prior to storage) exhibits a foam collapse time comparable to or less than that of the ASACOL® foam, such as a foam collapse time of greater than 5 minutes or a foam collapse time of less than 5 minutes, such as a foam collapse time of less than 4 minutes or less than 3 minutes.

Stability

In some embodiments, the foam formulations described herein exhibit good foam properties after storage, such as good expansion, cling, inversion, density, and collapse properties. In specific embodiments, one more of the expansion, inversion, cling, density, and collapse properties are substantially the same before and after storage, such as before and after storage for 14 days under accelerate conditions (40° C. and 70% relative humidity), such as exhibiting the same properties, or substantially the same properties differing by +/−20%, +/−10%, or less of the measured parameter (which may be measured as illustrated above).

Therapeutic Methods

As noted above, the foam formulations described herein can be used to rectally administer any drug, for any desired effect, including diagnostic, prophylactic, therapeutic, or cosmetic effect. In such methods, the formulation is typically is administered rectally from a dispenser specifically designed for that purpose. In some embodiments, a foam formulation is administered 1-3 times a day. In other embodiments, a foam formulation is administered once a day.

In specific embodiments, foam formulations described herein formulated with 5-ASA (or another aminosalicylate drug, or a pharmaceutically acceptable salt or ester of 5-ASA or other aminosalicylate drug) can be used rectally to treat inflammatory bowel disease, including ulcerative colitis or Crohn's disease. In some embodiments, a 5-ASA foam formulation as described herein is administered once or twice a day for a period of time of 4 to 6 weeks.

### EXAMPLES

The following specific examples are included as illustrative of the compositions described herein, using 5-ASA as a representative therapeutic agent. These example are in no way intended to limit the scope of the invention. Other aspects of the invention will be apparent to those skilled in the art to which the invention pertains.

The following procedures can be used to produce the foam formulations described in the examples below.

### Example 1

### Preparation of Anhydrous Foam Formulation

An anhydrous foam formulation was prepared using the following components:

| Formulation A | % W/W |
|---|---|
| Glycerin, USP | 4.00 |
| Xanthan Gum | 0.20 |
| PEG 400 | 25.00 |
| Emulsifying Wax (Polawax ™ NF) | 2.10 |
| Glyceryl Stearate SE | 0.60 |
| Sorbitan Oleate (Span ™ 80) | 0.80 |
| Petrolatum | 12.00 |
| Dimethyl Isosorbide | 2.50 |
| Caprylic/Capric Triglyceride | 2.90 |
| Methylparaben, NF | 0.10 |
| Propylparaben | 0.05 |
| BHA | 0.10 |
| BHT | 0.10 |
| Propylene Glycol, USP | 29.35 |
| Mesalamine | 20.00 |
| Tocopheryl Acetate | 0.20 |
| Total | 100.00 |

Glycerin is added to an appropriately sized side vessel and agitated using a propeller. Xanthan gum is sprinkled in and mixing is continued until well dispersed. The glycerin/xanthan gum premix is held until needed.

PEG 400 is added to a main mixing vessel and the vessel is charged with nitrogen to minimize exposure to air. Nitrogen flow is continued into the vessel at a low level along with propeller agitation. Mixing is continued throughout entire batch unless otherwise noted. While heating to 70-75° C., Polawax™NF (a self-emulsifying wax available from Croda, Inc), glyceryl stearate, Span 80 (sorbitan oleate), petrolatum, dimethyl isosorbide, caprylic/capric triglyceride, methylparaben, propylparaben, BHA and BHT are added with mixing between additions. Heating is continued to 70-75° C.

The glycerin/xanthan gum premix is added to the main mixing vessel and mixed for not less than 10 minutes.

Propylene glycol is then added under propeller agitation and the mixture is cooled to 35-40° C. When the batch temperature is less than 35° C. and there is an appropriate level of nitrogen in the main vessel, Mesalamine is sprinkled in, and mixing speed is increased as needed. Following the last addition, the mixture is homogenized for a minimum of 15 minutes or until uniform.

Tocopheryl acetate is added and mixing is continued for a minimum of 15 minutes.

The batch is allowed to rest at least 12 hours before filling, stored in a well-sealed container under a nitrogen blanket and protected from exposure to light.

For filling, the batch is mixed well under a nitrogen blanket and weighed into a dispenser, such as a 35×70 CCL (aluminum) can for dispensing for pharmaceutical foam compositions. The dispenser is vacuum crimped, gassed with propellant at a filling ratio of 90.0% formulation/10.0% propellant), and a dispensing valve (such as an S90 630 EQL valve) is put in place. Prior to filling with propellant, the dispenser may optionally be purged with nitrogen.

Additional anhydrous foam formulations as described below were prepared in accordance with the method described above.

| | % W/W | | |
|---|---|---|---|
| | Formulation | Formulation | Formulation |
| | B | C | D |
| Glycerin, USP | 4.000 | 4.000 | 4.000 |
| Xanthan Gum | 0.200 | — | — |
| Hydroxypropyl cellulose | — | 0.200 | — |
| Dextrin | — | — | 0.200 |
| PEG 400 | 25.000 | 25.000 | 25.000 |
| Emulsifying Wax | 1.925 | 1.925 | 1.400 |
| (Polawax ™ NF) | | | |
| Glyceryl Stearate SE | 0.550 | 0.550 | 0.400 |
| Sorbitan Oleate | 0.800 | 0.800 | 0.800 |
| (Span ™ 80) | | | |
| Petrolatum | 11.000 | 11.000 | 8.000 |
| Dimethyl Isosorbide | 2.500 | 2.500 | 2.500 |
| Caprylic/Capric Triglyceride | 2.900 | 2.900 | 2.900 |
| Methylparaben, NF | 0.100 | 0.100 | 0.100 |
| Propylparaben | 0.050 | 0.050 | 0.050 |
| BHA | 0.100 | 0.100 | 0.100 |
| BHT | 0.100 | 0.100 | 0.100 |
| Propylene Glycol, USP | 30.575 | 30.575 | 34.250 |
| Mesalamine | 20.000 | 20.000 | 20.000 |
| Tocopheryl Acetate | 0.200 | 0.200 | 0.200 |
| Total | 100.000 | 100.000 | 100.000 |

Using the formulations listed above six foam formulations were prepared using A31 and A46, respectively, as the propellant, at a filling ratio of 90.0% formulation/10.0% propellant.

### Example 2

### Preparation of Oil-in-Water Emulsion Foam Formulation

An oil-in-water emulsion foam formulation was prepared using the following components:

| | Formulation E (% W/W) |
|---|---|
| Premix | |
| Propylene Glycol, USP | 4.00 |
| Xanthan Gum | 0.20 |
| Main Mix | |
| Purified Water, USP | 47.95 |
| Methylparaben, NF | 0.15 |
| PEG 75 | 1.50 |
| Dimethyl Isosorbide | 1.00 |
| Tetrasodium EDTA | 0.20 |
| Support Mix | |
| Petrolatum | 18.0 |
| Cetyl Alcohol | 1.25 |
| Sorbitan Oleate (Span 80) | 0.50 |
| Polyethylene glycol 40 Stearate | 5.00 |
| Propylparaben | 0.05 |
| BHA | 0.10 |
| BHT | 0.10 |
| Active | |
| Mesalamine | 20.0 |
| Total | 100 |

Premix: Propylene Glycol is added to an appropriately sized side vessel and agitated using a propeller. Xanthan gum is sprinkled in and mixing is continued until well dispersed. The premix is held until needed.

Main mix: Purified water is added to a main mixing vessel. The vessel is charged with nitrogen head to minimize exposure to air. Nitrogen flow is continued into the vessel at a low level, and propeller agitation is commenced. Mixing is continued throughout the entire batch unless otherwise noted. The premix is added to the main mixing vessel and mixed for not less than 10 minutes.

Methylparaben, PEG-75, dimethyl isosorbide and tetrasodium EDTA are added to the main mixing vessel, and heated to 80-85° C. Mixing is continued at 80-85° C. until the support mix is added.

Support Mix: The Support Mix components (petrolatum, cetyl alcohol, Span 80, Polyethylene glycol 40 stearate, propylparaben, BHT and BHA) are added to a support vessel and heated to 83-88° C. under propeller agitation. When the Support Mix is at 83-88° C., it is slowly added to the main mixing vessel.

Mixing is continued in the main mixing vessel for 10 minutes at 80-88° C., then the mixture is cooled to 25° C. under propeller agitation and under nitrogen flow.

When the batch temperature is less than 35° C. and there is an appropriate level of nitrogen in the main vessel, mesalamine is sprinkled in, and mixing speed is increased as needed. When the batch temperature reaches 25° C. mixing is continued for an additional 10 minutes.

The batch is allowed to rest at least 12 hours before filling, stored in a well-sealed container with nitrogen blanket and protected from exposure to light.

For filling, the batch is mixed well under a nitrogen blanket and weighed into a dispenser, such as a 35×70 CCL (aluminum) can for dispensing for pharmaceutical foam compositions. The dispenser is vacuum crimped, gassed with propellant at a filling ratio of 92.0% formulation/8.0% propellant), and a dispensing valve (such as an S90 630 EQL valve) is put in place. Prior to filling with propellant, the dispenser may optionally be purged with nitrogen.

Additional oil-in-water emulsion foam formulations as described below were prepared in accordance with the method described above.

| | Formulation F (% W/W) |
|---|---|
| Premix | |
| Propylene Glycol, USP | 4.00 |
| Xanthan Gum | 0.20 |
| Main Mix | |
| Purified Water, USP | 47.95 |
| Methylparaben, NF | 0.15 |
| PEG 75 | 1.50 |
| Dimethyl Isosorbide | 1.00 |
| Tetrasodium EDTA | 0.20 |
| Support Mix | |
| Petrolatum | 10.00 |
| Cetyl Alcohol | 1.25 |
| Stearic acid | 2.50 |
| C12-15 alkyl benzoate | 8.00 |
| Sorbitan Oleate (Span 80) | 0.50 |
| Polyethylene glycol 40 Stearate | 2.50 |
| Propylparaben | 0.05 |
| BHA | 0.10 |
| BHT | 0.10 |
| Active | |
| Mesalamine | 20.00 |
| Total | 100 |

Using the formulation described above, two foam formulations were prepared using A31 and A46, respectively, as the propellant at a filling ratio of 92.0% formulation/8.0% propellant.

### Example 3

### Foam Property and Stability Studies

Foam properties of the foam formulations described above and the Asacol® foam formulation were assessed prior to storage (e.g., within 30 days of manufacture) (T0) or after storage for 14 days under accelerated conditions (40° C. (±2° C.) and 75% RH (±5% RH)) (14 Days Acc), in accordance with the methodology set forth above. Two propellants were evaluated for each formulation.

Anhydrous: A=Propellant is 10% w/w A-31; B=Propellant is 10% w/w A-46

Emulsion: A=Propellant is 8% w/w A-31; B=Propellant is 8% w/w A-46.

The results are set forth below.

| | | Anhydrous | | Anhydrous | | Anhydrous | | Anhydrous | |
|---|---|---|---|---|---|---|---|---|---|
| | | (Form. A) | | (Form. B) | | (Form. C) | | (Form. D) | |
| | Asacol ® | A | B | A | B | A | B | A | B |
| Vol. of expansion (mL) | | | | | | | | | |
| T0 | 5.0 | 17.7 | 22.9 | 27.3 | 19.4 | 24.6 | 22.8 | 30.2 | 30.1 |
| 14 Days Acc | 13.9 | 18.5 | 21.4 | 19.4 | 20.8 | 19.0 | 25.6 | 27.8 | 27.3 |
| Foam Cling (cm/g) | | | | | | | | | |
| T0 | 7.5 | 0.5 | 2.8 | 2.1 | 2.4 | 2.6 | 1.9 | 3.0 | 2.8 |
| 14 Days Acc | 13.0 | 1.0 | 0.6 | 2.1 | 1.9 | 4.0 | 5.9 | 3.7 | 4.6 |
| Foam Density (g/mL) | | | | | | | | | |
| T0 | 0.067 | 0.110 | 0.109 | 0.113 | 0.106 | 0.119 | 0.106 | 0.115 | 0.098 |
| 14 Days Acc | 0.070 | 0.114 | 0.112 | 0.112 | 0.103 | 0.130 | 0.118 | 0.121 | 0.109 |
| Foam Collapse (37° C.) | | | | | | | | | |
| (min) | | | | | | | | | |
| T0 | >5 | >5 | 3.5 | 3.5 | 4.1 | 3.0 | 3.1 | >5 | 2.4 |
| 14 Days Acc | >5 | 3.4 | 4.1 | 2.6 | 3.4 | 2.3 | 3.3 | 2.6 | 3.3 |
| Foam Inversion (min) | | | | | | | | | |
| T0 | 1.6 | >20 | >20 | >20 | >20 | 4.2 | >20 | 4.0 | 1.8 |
| 14 Days Acc | 9.3 | >20 | >20 | >20 | >20 | >20 | >20 | 4.8 | 6.8 |

| | |
|---|---|
| Emulsion | Emulsion |
| (Form. E) | (Form. F) |

| | Asacol ® | A | B | A | B |
|---|---|---|---|---|---|
| Volume of expansion | | | | | |
| (mL) | | | | | |
| T0 | 5.0 | 30.3 | 27.0 | 39.9 | 38.3 |
| 14 Days Acc | 13.9 | 32.1 | 34.5 | 37.0 | 37.0 |
| Foam Cling (cm/g) | | | | | |
| T0 | 7.5 | 3.4 | 2.6 | 7.5 | 2.6 |
| 14 Days Acc | 13.0 | 0.7 | 3.8 | 0.9 | 1.5 |
| Foam Density (g/mL) | | | | | |
| T0 | 0.067 | 0.056 | 0.051 | 0.068 | 0.058 |
| 14 Days Acc | 0.070 | 0.058 | 0.052 | 0.060 | 0.062 |
| Foam Collapse (37° C.) | | | | | |
| (min) | | | | | |
| T0 | >5 | >5 | >5 | >5 | >5 |
| 14 Days Acc | >5 | >5 | >5 | >5 | >5 |
| Foam Inversion (min) | | | | | |
| T0 | 1.6 | >20 | >20 | >20 | >20 |
| 14 Days Acc | 9.3 | >20 | >20 | >20 | >20 |

The stability studies showed that all examples maintained suitable properties, such that amounts of components stayed within the 90-110% range.

### Example 4

### Robustness Studies

Anhydrous Formulation

Formulation A described in Example 1 above was used as a reference formulation for an anhydrous foam formulation robustness study. The formulation was varied as indicated in the table below and properties of the resulting formulation were assessed.

| | | Parameter | | | | |
|---|---|---|---|---|---|---|
| | | | Volume of | Foam | Foam | Foam |
| Formulation | | Viscosity | Expansion | Density | Break | Cling |
| Number | Variation | (cps) | (mL) | (g/mL) | (min) | (cm/g) |
| Anhydrous | — | 7500 | 21.8 | 0.121 | 4.1 | 3.78 |
| Formulation A | | | | | | |
| A-1 | Increase | 4500 | 19.7 | 0.124 | 4.7 | 3.54 |
| | Polawax ™ NF by | | | | | |
| | 5% | | | | | |
| A-2 | Decrease | 5000 | 16.5 | 0.135 | 3.6 | 4.07 |
| | Polawax ™ NF by | | | | | |
| | 5% | | | | | |
| A-3 | Increase Span ™ | 9500 | 22.0 | 0.130 | 4.4 | 2.92 |
| | 80 by 5% | | | | | |
| A-4 | Decrease Span ™ | 6000 | 19.7 | 0.130 | 4.3 | 3.86 |
| | 80 by 5% | | | | | |
| A-5 | Increase | 9500 | 21.3 | 0.125 | 4.4 | 3.50 |
| | Petrolatum by 5% | | | | | |
| A-6 | Decrease | 5000 | 20.3 | 0.127 | 4.8 | 4.10 |
| | Petrolatum by 5% | | | | | |
| A-7 | Increase A-31 | 7500 | 19.5 | 0.104 | 4.5 | 3.51 |
| | Propellant by 20% | | | | | |
| A-8 | Decrease A-31 | 7500 | 22.6 | 0.133 | 4.5 | 3.08 |
| | Propellant by 20% | | | | | |
| A-9 | Lower mixing | 5000 | 19.2 | 0.128 | 4.9 | 3.47 |
| | temperature: | | | | | |
| | Main Vessel, mix | | | | | |
| | at 60-65° C. | | | | | |
| A-10 | Rapid Addition of | 9000 | 16.0 | 0.128 | 5.0 | 2.69 |
| | Xanthan Gum to | | | | | |
| | Premix IA and of | | | | | |
| | Premix 1 A to | | | | | |
| | Main Vessel | | | | | |

Emulsion Formulation

Formulation E described in Example 2 above was used as a reference formulation for an emulsion foam formulation robustness study. The formulation was varied as indicated in the table below and properties of the resulting formulation were assessed.

| | | Parameter | | | | | |
|---|---|---|---|---|---|---|---|
| | | | | Volume of | Foam | Foam | Foam |
| Formulation | | Viscosity | | Expansion | Density | Break | Cling |
| Number | Variation | (cps) | pH | (mL) | (g/mL) | (min) | (cm/g) |
| Emulsion | | 8000 | 5.84 | 25.5 | 0.062 | >5 | 2.89 |
| Formulation E | | | | | | | |
| E-11 | Increase | 10000 | 5.81 | 26.3 | 0.059 | >5 | 3.67 |
| | Petrolatum | | | | | | |
| | by 5% | | | | | | |
| E-12 | Decrease | 8000 | 5.84 | 24.0 | 0.062 | >5 | 4.59 |
| | Petrolatum | | | | | | |
| | by 5% | | | | | | |
| E-13 | Increase PEG 40 | 6500 | 5.84 | 27.7 | 0.061 | >5 | 4.60 |
| | Stearate by 5% | | | | | | |
| E-14 | Decrease PEG | 9000 | 5.86 | 23.3 | 0.059 | >5 | 5.23 |
| | 40 Stearate by | | | | | | |
| | 5% | | | | | | |
| E-15 | Increase A-31 | 8000 | 5.84 | 30.9 | 0.075 | >5 | 5.09 |
| | Propellant | | | | | | |
| | by 20% | | | | | | |
| E-16 | Decrease A-31 | 8000 | 5.84 | 15.3 | 0.063 | >5 | 5.10 |
| | Propellant | | | | | | |
| | by 20% | | | | | | |
| E-17 | Eliminate | 8000 | 4.25 | 23.0 | 0.063 | >5 | 5.52 |
| | Tetrasodium | | | | | | |
| | EDTA | | | | | | |
| E-18 | Increase | 8500 | 6.17 | 25.3 | 0.063 | >5 | 5.52 |
| | Tetrasodium | | | | | | |
| | EDTA by 100% | | | | | | |
| E-19 | Lower mixing | 10000 | 5.80 | 24.2 | 0.063 | >5 | 4.98 |
| | temperature: | | | | | | |
| | Main Vessel, | | | | | | |
| | mix at 70-75° C. | | | | | | |
| E-20 | Rapid Addition | 8500 | 5.75 | 15.3 | 0.059 | >5 | 4.48 |
| | of Support Mix | | | | | | |
| | to Main Vessel | | | | | | |

Based on the results, the following components were determined to impact the following parameters:

| | | Parameter Impacted | | | | | |
|---|---|---|---|---|---|---|---|
| | Primary | | | Volume of | Foam | Foam | Foam |
| Component | Function | Viscosity | pH | Expansion | Density | Break | Cling |
| Petrolatum | Thickener | X | | X | X | X | X |
| Polyoxyl 40 | Thickener | X | | X | X | X | X |
| Stearate | Emulsifier | | | | | | |
| A-31 Propellant | Dispensing | | | X | X | X | X |
| Tetrasodium | Chelating | X | X | X | X | X | X |
| EDTA | Agent | | | | | | |
| Polawax ™ | Thickener | X | | X | X | X | X |
| Span ™ 80 | Emulsifier | X | | X | X | X | X |
| Lower mixing | Process | X | | X | X | X | X |
| temperature: | | | | | | | |
| (Anhydrous) | | | | | | | |
| Rapid Addition | Process | X | | X | X | X | X |
| (Anhydrous) | | | | | | | |
| Lower mixing | Process | X | | X | X | X | X |
| temperature: | | | | | | | |
| (Emulsion) | | | | | | | |
| Rapid Addition | Process | X | | X | X | X | X |
| (Emulsion) | | | | | | | |

### Example 5

### Ex Vivo Studies

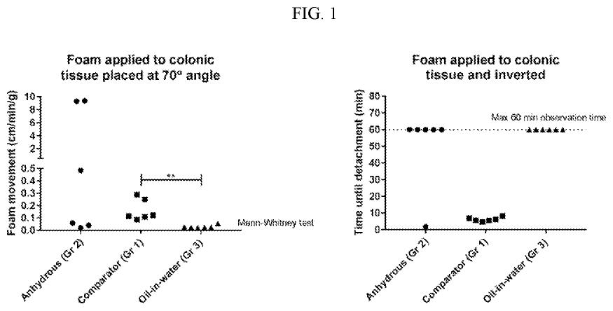

Formulation A described in Example 1 and Formulation E described in Example 2 were tested in ex vivo foam cling and foam inversion studies using pig colonic mucosa, using ASACOL® foam as a comparator. Results are reported in FIG. 1.

In the foam cling test (left panel), Formulation E (“Oil-in-water”) travelled the shortest distance per unit time per gram. This value was significantly lower than for the ASACOL® foam (“Comparator”), according to the Mann-Whitney test. For Formulation A (“Anhydrous”), three of the six tested replicates had lower foam movement than the comparator foam, whereas two outliers that moved quickly down the colon tissue were observed. In the foam inversion test (right panel), which involved application to the intestinal colonic surface and inversion, both test formulations showed better adhesion than the comparator formulation.

The results show that formulations described herein exhibit increased adhesion after application to a relevant biological surface (colon tissue) as compared to ASACOL® foam.

What is claimed is:

1. An anhydrous pharmaceutical rectal foam formulation comprising an aminosalicylate drug or a pharmaceutically acceptable salt or ester thereof, a waxy/lipid component, a solvent/carrier component, an emulsifier/surfactant component, a gum/resin component, and a propellant.

2. The anhydrous formulation of claim 1, wherein the waxy/lipid component comprises one or more components selected from the group consisting of petrolatum, cetyl alcohol, stearyl alcohol, caprylic/capric triglycerides (CCTs), caprylic/capric glycerides, and sodium stearate.

3. The anhydrous formulation of any one of claims 1-2, comprising about 5-15% w/w of the waxy/lipid component.

4. The anhydrous formulation of any one of claims 1-3, wherein the solvent/carrier component comprises one or more components selected from the group consisting of polyethylene glycol 400, glycerin, propylene glycol, and mineral oil.

5. The anhydrous formulation of any one of claims 1-4, comprising about 55-65% w/w of the solvent/carrier component.

6. The anhydrous formulation of any one of claims 1-5, wherein the emulsifier/surfactant component comprises one or more components selected from the group consisting of glyceryl isostearate, emulsifying wax, glyceryl stearate, glyceryl tristearate, sorbitan oleate, polyglyceryl-3 laurate and polyglyceryl-3 diisostearate.

7. The anhydrous formulation of any one of claims 1-6, comprising about 5-25% w/w of the emulsifier/surfactant component.

8. The anhydrous formulation of any one of claims 1-7, wherein the gum/resin component comprises one or more components selected from the group consisting of methylcellulose, hydroxypropyl cellulose, xanthan gum, pectin, and dextrin.

9. The anhydrous formulation of any one of claims 1-8, comprising about 0.1-2% w/w of the gum/resin component.

10. The anhydrous formulation of any one of claims 1-9, comprising about 20% w/w 5-aminosalicylic acid (5-ASA), about 25% w/w polyethylene glycol 400, about 4% w/w glycerin, about 29.35% w/w propylene glycol, about 2.9% w/w CCTs, about 2.1% w/w emulsifying wax, about 0.6% w/w glyceryl stearate, about 0.8% w/w sorbitan oleate, about 12% w/w petrolatum, about 0.2% w/w xanthan gum, and about 10% w/w propellant A31.

11. An oil-in-water emulsion pharmaceutical rectal foam formulation comprising an aminosalicylate drug or a pharmaceutically acceptable salt or ester thereof, a solvent/carrier component comprising water, an emulsifier/surfactant component, a waxy/lipid component, a gum/resin component, and a propellant.

12. The emulsion formulation of claim 11, wherein the solvent/carrier component comprises water and one or more components selected from the group consisting of polyethylene glycol 400, glycerin, propylene glycol and C₁₂-C₁₅ alkyl benzoates.

13. The emulsion formulation of any one of claims 11-12, comprising about 30-50% w/w water and about 4% w/w propylene glycol.

14. The emulsion formulation of any one of claims 11-13, wherein the emulsifier/surfactant component comprises one or more components selected from the group consisting of polyethylene glycol 75, polyethylene glycol 40 stearate, and sorbitan oleate.

15. The emulsion formulation of any one of claims 11-14, comprising about 2-25% w/w of the emulsifier/surfactant component.

16. The emulsion formulation of any one of claims 11-15, wherein the waxy/lipid component comprises one or more components selected from the group consisting of petrolatum, cetyl alcohol, caprylic/capric triglycerides (CCTs), caprylic/capric glycerides, and stearic acid.

17. The emulsion formulation of any one of claims 11-16, comprising about 10-25% w/w of the waxy/lipid component.

18. The emulsion formulation of any one of claims 11-17, wherein the gum/resin component comprises one or more components selected from the group consisting of hydroxyethyl cellulose and xanthan gum.

19. The emulsion formulation of any one of claims 11-18, comprising about 0.1-0.2% w/w of the gum/resin component.

20. The emulsion formulation of any one of claims 11-19, comprising about 20% w/w 5-aminosalicylic acid (5-ASA), about 47.95% w/w water, about 4% w/w propylene glycol, about 1.5% w/w polyethylene glycol 75, about 5% w/w polyethylene glycol 40 stearate, about 0.5% w/w, about 18% w/w petrolatum, about 1.25% w/w cetyl alcohol, about 0.2% w/w xanthan gum, and about 8% w/w propellant A31.

21. The formulation of any one of claims 1-20, wherein the aminosalicylate drug is 5-ASA or a pharmaceutically acceptable salt or ester thereof.

22. The formulation of any one of claims 1-20, comprising from about 5 w/w to about 30%w/w 5-ASA.

23. The formulation of any one of claims 1-22, further comprising a propellant.

24. The formulation of claim 23, comprising about 5-15% w/w propellant.

25. The formulation of any one of claims 22-23, wherein the propellant is one or more of propellant A-31 and propellant A-46.

26. The formulation of any one of claims 1-25, further comprising a penetration enhancer.

27. The formulation of claim 26, wherein the penetration enhancer comprises dimethyl isosorbide.

28. The formulation of any one of claims 1-27, further comprising one or more additional components selected from the group consisting of pH adjusting agents, antioxidants, and preservatives.

29. A foam made from a formulation according to any one of claims 1-28.

30. The foam of claim 29, wherein the foam exhibits a volume of expansion of at least 15 mL in 5 minutes, when tested according to monograph 1105 of the European Pharmacopoeia.

31. The foam of any one of claims 29-30, wherein the time to foam collapse in the foam collapse test is at least 3 minutes.

32. The foam of any one of claims 29-31, wherein the time to foam dislodgement in the foam inversion test is at least 20 minutes.

33. The foam of any one of claims 29-32, wherein the foam exhibits a foam cling of no more than 6 cm/g per 1-2 g of foam over a period of 5 mins.

34. The foam of any one of claims 29-33, wherein the foam is made from an anhydrous formulation and exhibits a foam density of at least 0.10 g/mL.

35. The foam of any one of claims 29-33, wherein the foam is made from an emulsion formulation and exhibits a foam density of at least 0.05 g/mL.

36. A method of making an anhydrous formulation of any one of claim 1-10 or 21-28, comprising:
(a) preparing a first mixture of a solvent/carrier component, a waxy/lipid component and an emulsifier/surfactant component;
(b) adding a gum/resin component to the first mixture;
(c) adding the aminosalicylate drug or pharmaceutically acceptable salt or ester thereof to the mixture (b) to form a final mixture; and
(d) combining the final mixture with a propellant.

37. A method of making an emulsion formulation of any one of claims 11-28, comprising:
(a) preparing a first mixture of a solvent/carrier component comprising water and a gum/resin component;
(b) preparing a second mixture of an emulsifier/surfactant component and a waxy/lipid component;
(b) combining the first and second mixtures to obtain a third mixture;
(c) adding the aminosalicylate drug or pharmaceutically acceptable salt or ester thereof to the third mixture to form a final mixture; and
(d) combining the final mixture with a propellant.

38. A method of administering an aminosalicylate drug to a subject in need thereof, comprising rectally administering a formulation according to any one of claims 1-28 or a foam according to any one of claims 29-35.

39. The method of claim 38, wherein the formulation or foam comprises 5-ASA and is administered from an aerosol dispenser dispensing an amount of formulation providing from about 1 g to about 5 g 5-ASA per dose.

40. A method of treating inflammatory bowel disease in a subject in need thereof, comprising rectally administering a formulation according to any one of claims 1-28 or a foam according to any one of claims 29-35 to the subject.

41. The method of claim 40, wherein the inflammatory bowel disease is selected from ulcerative colitis and Crohn's disease.

42. A formulation according to any one of claims 1-28 or a foam according to any one of claims 29-35, for administering an aminosalicylate drug to a subject in need thereof.

43. A formulation according to any one of claims 1-28 or a foam according to any one of claims 29-35, for treating an inflammatory bowel disease in a subject in need thereof.

44. The formulation or foam of claim 43, wherein the inflammatory bowel disease is selected from ulcerative colitis and Crohn's disease.

45. Use of a formulation according to any one of claims 1-28 or a foam according to any one of claims 29-35 in the preparation of a medicament for administering an aminosalicylate drug to a subject in need thereof.

46. Use of a formulation according to any one of claims 1-28 or a foam according to any one of claims 29-35 in the preparation of a medicament for treating an inflammatory bowel disease in a subject in need thereof.

47. The use of claim 46, wherein the inflammatory bowel disease is selected from ulcerative colitis and Crohn's disease.
